(19) United States
(12) Patent Application Publication
(10) Pub. No.: US 2005/0048141 A1
(43) Pub. Date: Mar. 3, 2005

(54) Use of the plant Cissus quadrangularis or its extracts for osteo-health including prevention or mitigation of degenerative disease, fracture healing and anabolic increase in osteo tissues

(76) Inventors: Brian Paul Cortes, Lawndale, CA (US); Lal Hingorani, Khan West (IN); Vijay Thawani, Clarke Town (IN)

Correspondence Address: Shannon McGarrah;Amin Law, LLC, Ste. 500, 217 N. Jefferson St., Chicago, IL, 60661, US

(21) Appl. No.: 10/927,727

(22) Filed: Aug. 27, 2004

Related U.S. Application Data
(60) Provisional application No. 60/498,955, filed on Aug. 30, 2003.

(51) Int. Cl. A61K 35/78

(52) U.S. Cl. 424/725

(57) ABSTRACT

The present invention relates to a composition containing Cissus quadrangularis from the family Cissus sp. or its extracts for use in a nutritional product, dietary supplement or pharmaceutical composition wherein the composition provides prevention and treatment of osteo-health related disorders and diseases as well as effective fracture healing through the use of anabolic steroid properties that increase the therapeutic process. The invention also relates to an extraction process for obtaining such composition.

### FIELD OF INVENTION

The present invention relates to a composition for use in a nutritional product, dietary supplement or pharmaceutical composition wherein the composition provides therapeutic use for osteo-health, including prevention or mitigation of degenerative disease, fracture healing and anabolic increase in osteo tissues. More particularly, the present invention relates to a composition containing Cissus quadrangularis from the family Cissus sp. or its extracts, and an extraction process for obtaining such composition.

### BACKGROUND OF THE INVENTION

Osteoporosis is a systemic skeletal disease characterized by low bone mass and micro architectural deterioration of bone tissue, with a consequent increase in bone fragility and susceptibility to fracture. Osteoporosis results mainly in not having enough bone tissue, which is referred to as low bone density or low bone mass. The loss of bone density and structural weakness leads to bones that can break under the slightest strain. The prevalence of low bone mass density increases with age. As the number of people in middle and old age rises, this potentially debilitating disease becomes an increasingly important cause of painful fractures and consequences like deformity, loss of mobility and even death. The major osteoporotic complications are fractures of the hip, spine and Colle's fracture.

Quality of life is greatly impaired in persons with severe osteoporosis, not only because of pain and deformity, but also because of the limited ability of movement and the fear of breaking more bones. The physical debility after fracture healing may lead to severe impairment in the quality of life.

Other modes of therapy include calcium supplementation, Hormone Replacement Therapy (HRT) for postmenopausal osteoporosis, and other treatment modalities like Bisphosphonates. The use of calcium as a supplement in clinical trials evaluating calcium and vitamin D are inconsistent with some supplements demonstrating benefits and others not indicating clear utility. HRT reduces the risk of spine and hip fractures with sufficient dosage but the benefits associated with HRT are offset by side effects like breast cancer, thromboembolism, and withdrawal bleeding. Drawbacks commonly associated with the use of Bisphosphonates include esophagitis, diarrhea and constipation. The use of these types of therapies and their associated side effects and drawbacks indicate a need for other options in the prevention and treatment of osteoporosis and promotion of increased osteo health.

Interest in herbal medicines has increased because these are considered to be relatively safer than chemically synthesized drugs. India has used many traditional Ayurvedic medicines for centuries. One such Ayurvedic herbal medicine is Cissus quadrangularis. Cissus quadrangularis is an indigenous medicinal plant of India. The use of this plant for promoting fracture healing has been known for many centuries but the effects on total osteo-health and related disorders, including prevention and mitigation of degenerative diseases such as osteoporosis, as well as the anabolic increase observed in fracture healing, has heretofore not been explored with a high potency extract.

Thus, there is a need and a demand for a beneficial compound containing Cissus quadrangularis that is a high potency extract. In particular, there is a need and a demand for a high potency extract that demonstrates the intended features of this invention; namely, the prevention and treatment of osteo-health related disorders and diseases combined with effective fracture healing through the use of anabolic steroid properties that increase the therapeutic process.

### SUMMARY OF THE INVENTION

A general object of this invention is to provide a beneficial compound that is extracted from a natural source for use in a nutritional product, dietary supplement or pharmaceutical composition.

A more particularized object of this invention is to provide a high potency composition derived from the plant family Cissus sp.

The general purpose of this composition is to maintain and restore bone strength safely and effectively. Increase in bone mineral density has been shown to decrease future fracture risk.

A more particularized purpose of this composition is to provide generalized fracture healing through the use of the composition and the action of the composition's anabolic steroidal properties on osteo tissues.

The prior art has generally failed to provide a high potency composition derived from the plant family Cissus sp. or its extracts that is effective for osteo health, specifically for the prevention and mitigation of degenerative diseases related to osteo health, for use in fracture healing and anabolic increase in osteo tissues. The prior art has also failed to provide a method for producing a high potency composition derived from the plant family Cissus sp.

### DETAILED DESCRIPTION OF THE INVENTION

The present invention provides a beneficial compound for use in a nutritional product, dietary supplement or pharmaceutical composition that is derived from the plant family Cissus sp. or its extracts. The present invention also provides an extraction method for producing a composition derived from the plant family Cissus sp.

Cissus quadrangularis is an indigenous medicinal plant of India. The Latin name is Cissus quadrangula Linn and is also known as Vitis quadrangularis Wall. Other synonyms include Vajravalli, Chaturdhara, Harjara, Hadajora, Hadasnakala, Hadjod, Mangaraballi, Changalam Parande, Kandvel, Hadbhanga, Haddjor, Perandai, Nalleru and Hathjod.

This invention concerns extracts of the plant family Cissus sp. for use in the prevention or mitigation of degenerative diseases related to osteo health, use in fracture healing and promotion of anabolic increases in osteo tissues. Cissus quadrangularis has been shown to contain calcium oxalate, carotene, ascorbic acid, vitamin C, and anabolic steroidal substances. These anabolic steroidal principles from Cissus quadrangularis showed a marked influence in the rate of fracture healing by influencing early regeneration of all connective tissues of mesenchyma origin, namely the fibroblasts, the chondroblasts and osteoblasts involved in the healing and quicker mineralization of the callus. The probable mechanism of action in fracture healing is believed in part to be due to the stimulation of the metabolism and increased uptake of the calcium, sulfur, and strontium by the osteoblasts.

Cissus quadrangularis is shown to neutralize the anti-anabolic effect of steroid-like cortisone in the healing of fractures. Antianabolic effects of cortisone include inhibition of tissue regeneration and repair, and additionally retarding formation of specific skeletal structures.

Imbalance in the activities of osteoclasts (cells responsible for bone loss) and osteoblasts (cells responsible for bone formation) may lead to osteoporosis and fractures in postmenopausal women. In osteoporosis, the bones begin to deteriorate due to calcium deficiency as a result of the body's efficiency in maintaining mineral balance in the blood at the expense of bone integrity. Cissus quadrangularis has shown significant ability to inhibit antianabolic effects and bone fracture healing effects likely to exert some beneficial effects on recovery of bone mineral density in postmenopausal osteoporosis. The purpose of this invention is to maintain and to restore bone strength safely and effectively, and to prevent or decrease degenerative diseases and disorders relating to osteo health by increasing bone mineral density in the osteo tissues. Increase in bone mineral density has been shown to decrease future fracture risk. Further, the purpose of this invention is to provide fracture healing through the use of the anabolic steroidal properties of the plant and its extracts. The active ingredient in Cissus quadrangularis is attributed to the presence of a phytogenic steroid that plays a major role in fracture healing and has anabolic properties. The anabolic phytosteroid has been shown to markedly induce an early regeneration of the connective tissue and rapid mineralization of the callus. This steroid fracture also has androgenic properties.

One preferred embodiment of the present invention is directed to a composition comprising an extract of the plant from the family Cissus sp. for use in a nutritional product, dietary supplement or pharmaceutical composition. Suitably, the extract from the family Cissus sp. is Cissus quadrangularis. Generally, the extract may be prepared in any dosage form suitable for oral administration.

A preferred embodiment of the present invention is directed to a process for making a composition comprising an extract of the plant from the family Cissus sp. for use in a nutritional product, dietary supplement or pharmaceutical composition. Suitably, the extract obtained from the process may be derived from an alcohol-based extraction process. Alternatively, the extract obtained from the process may be derived from an aqueous-based solution.

Generally, the extract composition is obtained by: (1) providing plant component part of the family Cissus sp.; (2) extracting the plant component part substantially immediately; (3) concentrating the extract by filtering, discarding the residue and collecting the extract concentrate as a paste; (4) vacuum drying the paste to form a dry powder; and (5) pulverizing and sifting the powder to form the product.

### EXAMPLE 1

Plant from the Cissus sp. Family:

Preparation Method:

1. Charge crushed Cissus quadrangularis in an S.S vessel;
2. Add ethanol;
3. Reflux at 80° C. to 90° C. for approximately 3 hours;
4. Filter the ethanol extract solution using a filter press;
5. Collect the ethanol extract solution in an S.S vessel;
6. Concentrate the ethanol extract to a thick paste;
7. Dry the thick past in a vacuum dryer; and
8. Pulverize and sift the material.

While in the foregoing specification this invention has been described in relation to certain preferred embodiments thereof, and many details have been set forth for the purpose of illustration, it will be apparent to those skilled in the art that the invention is susceptible to additional embodiments and that certain details described herein can be varied considerably without departing from the basic principles of the invention.

1. A composition comprising an extract of the plant from the family Cissus sp. for use in a nutritional product, dietary supplement or pharmaceutical composition.

2. The composition of claim 1, wherein the extract from the Cissus sp. family is Cissus quadrangularis.

3. The composition of claim 1, wherein the extract has been obtained by an alcohol-based extraction process.

4. The composition of claim 1, wherein the extract has been obtained by an aqueous-based extraction process.

5. The composition of claim 1, wherein the extract is present in a dosage form suitable for oral administration.

6. A process of making the composition of claim 1 which comprises: (1) providing plant component part of the Cissus sp. family; (2) extracting the plant component part substantially immediately; (3) concentrating the extract by filtering, discarding the residue and collecting the extract concentrate as a paste; (4) vacuum drying the paste to form a dry powder; and (5) pulverizing and sifting the powder to form the product.

7. The process of making the composition of claim 6 wherein the plant component part of the Cissus sp. family is Cissus quadrangularis.

8. The process of claim 6 wherein the extraction of the plant component part is with an alcohol solution.

9. The process of claim 6 wherein the extraction of the plant component part is with an aqueous solution.
